(19) United States
(12) Patent Application Publication
(10) Pub. No.: US 2004/0197905 A1
(43) Pub. Date: Oct. 7, 2004

(54) Methods and devices for monitoring cellular metabolism in microfluidic cell-retaining chambers

(75) Inventor: Dean G. Hafeman, Hillsborough, CA (US)

Correspondence Address: TESTA, HURWITZ & THIBEAULT, LLP, HIGH STREET TOWER, 125 HIGH STREET, BOSTON, MA, 02110, US

(73) Assignee: Thermogenic Imagining, Billerica, MA

(21) Appl. No.: 10/759,374

(22) Filed: Jan. 16, 2004

Related U.S. Application Data
(60) Provisional application No. 60/440,571, filed on Jan. 16, 2003.

(51) Int. Cl. C12N 5/00; C12N 5/02

(52) U.S. Cl. 435/374; 435/289.1

(57) ABSTRACT

A system is provided to enable maintaining cell viability in a microfluidic device and for monitoring an activity of a cell in a microfluidic device. The microfluidic device may include a cell duct plate, a flow channel plate, a porous membrane bounding at least a portion of each of the cell duct and the flow channel plate. The system may include, in addition to the microfluidic device, a pump, a controller, and a sensor.

### RELATED APPLICATIONS

This application claims the benefit of U.S. Provisional Application Serial No. 60/440,571, filed on Jan. 16, 2003, the entire disclosure of which is hereby incorporated by reference.

### FIELD OF THE INVENTION

This application relates generally screening techniques and, more specifically, to the monitoring of nutrient consumption by living cells and of cell products.

### BACKGROUND

In the study of biological process there is frequently a need to monitor a concentration of metabolic nutrients consumed (or products produced) by organisms, tissues, or cells during a known time interval. The metabolic rate of nutrient consumption or production may be established by dividing the change in concentration of nutrient or product by the time interval over which the concentration change occurs. For studying such processes in single cells or small number of cells it is advantageous for the cells to be contained in a small metabolic chamber so that the change in concentration of such nutrients or products are large enough to be measured. The term “microphysiometer” has been used for a device that monitors the rate of extracellular acidification of nutrient medium surrounding cells in a micro-metabolic chamber. See, for example, J. W. Parce et al. (1989) “Detection of Cell Affecting Agents with a Silicon Biosensor.” Science 246: 243; Owicki, J. C., Bousse, L. J., Hafeman, D. G., Kirk, G. L., Olson, J. D., Wada, H. G. and Parce, J. W. (1994) “The Light-Addressable Potentiometric Sensor: Principles and Biological Applications.” Ann. Rev. Biophys. Biomol. Struct. 23: 87; and McConnell, H. M., Owicki, J. C., Parce, J. W., Miller, D. L., Baxter, G. T., Wada, H. G. and Pitchford, S. (1992) “The Cytosensor Microphysiometer: Biological Applications of Silicon Technology” Science 257: 1906 (and the references contained therein); these references are hereby incorporated by reference.

Although limited to pH measurements, such microphysiometer devices known in the prior art nevertheless have found useful application. Notable uses have been in the search for drug candidates (e.g., receptor agonists, antagonists, chemokines, growth factors, toxic compounds, etc.). Particularly useful is the ability to perform continuous (i.e., real-time) measurements following treatment of cells with potential drug candidates. See, for example, Wada, H. G. Indelicato, S. R., Meyer, L. Kitamura, T., Miyajima, A., Kirk, G., Muir, V. C. and Parce, J. W. (1993) “GM-CSF Triggers a Rapid Glucose Dependent Extracellular Mediated Activation of Acid Production.” J. Cell Physiol. 154: 129, hereby incorporated by reference. Another important use has been as a diagnostic aid in the selection of clinically effective cytostatic drugs in treatment of cancer. See for example, Metzgar, R., Deglmann, C. J., Hoerrlein, S., Zapf, S. and Hilfrich, J. (2001) Toxicology 166, 97-108, hereby incorporated by reference.

Although prior art microphysiometer devices, such as those mentioned above, have been found quite useful, unmet needs have limited their general use. Ideally, microphysiometer devices should:

a) Provide for more convenient use (e.g., have simple, inexpensive, and disposable components),

b) Provide for higher throughput (multiple samples may need to be determined in parallel),

c) Provide for a multiplicity of selectable cellular nutrients or metabolites that may be analyzed (i.e., more than just pH measurements are desired), and

d) Require fewer cells for an analysis in order to i) reduce the cost of obtaining or growing cells and ii) be able to acquire sufficient data with limited number of primary cells available directly from human patients.

### SUMMARY

In an aspect, the invention features a method for maintaining cell viability in a microfluidic device. The method includes providing a cell proximate a first side of a porous membrane of the microfluidic device, and providing a media comprising a cell nutrient proximate a second side of the porous membrane. The porous membrane is adapted to prevent the cell from passing therethrough, to substantially prevent the media from flowing therethrough, and to provide diffusive communication between the two sides to allow the cell nutrient and a cell product to pass therethrough.

One or more of the following features may be included. The method may include the step of detecting the cell product in the media. Detecting the cell product in the media may include detecting at least one of an electrochemical signal and a luminescent emission. The porous membrane may include a material such as glass fiber, polycarbonate, polyethylene, polypropylene, polystyrene, polyimide, cellulose, nitrocellulose, cellulose esters, nylon, rayon, fluorocarbon, perfluorocarbon, polydimethylsioloxane, polyester, acrylics, acrylonitrile-butadiene-styrene; polyoxy-methylene; polyarylate, polyvinylchloride, PBT-Polyester, polybenzimidazone, acetal copolymers, polyimides, ethylene-chlorotrifluorethylene, PET polyesters, ethylene-tetrafluorethylene, fluorinated ethylene propylene, polyphenylene sulfide, polyethylene, polyurathanes, polyketones, polychloro-trifluoro-ethylene, polyvinylidene fluoride, polyethylene terephthalate polyesters, polypropylene oxides, polypropylene styrenes, polyether-ether ketones, polytetrafluorethylene, polyarylether sulfones, polyamide-imides, polyphenylene sulfides, polyarylates, polymethylpentene, polyketones, polysulfones, polyphenylene sulfides, PBT polyesters, and/or alloys of polymers.

Providing the media may include flowing the media along at least a portion of the second side of the porous membrane, e.g., intermittently flowing the media. The method may include controlling a temperature of the cell. The method may include controlling a concentration of the cell nutrient in the media.

In another aspect, the invention features a method for loading cells into a microfluidic device. The method includes depositing a cell sample into a common duct opening of the microfluidic device, and subdividing the cell sample, so that at least a first portion of the sample flows into a first cell duct in fluidic communication with the duct opening and another portion of the sample flows into a second cell duct in fluidic communication with the duct opening.

One or more of the following features may be included. The step of subdividing the cell may include flowing at least a portion of the cell sample through a manifold interdisposed between the duct opening and at least one cell duct. At least one of the sample portions may flow by capillary action. The step of subdividing the cell sample may include substantially uniformly dividing the cell sample and/or applying a pressure differential. The cell sample may include a substantially isopycnic solution having a density substantially similar to a density of cells in the sample, such that the cells remain substantially in neutral suspension in the isopycnic solution.

In another aspect, the invention features a microfluidic device for maintaining viability of a cell. The microfluidic device includes a cell duct plate, defining at least one cell duct therein, a porous membrane having a first side bounding at least a portion of the cell duct, and a flow channel plate, defining at least one flow channel therein, at least a portion of the flow channel being bounded by a second side of the porous membrane, wherein the cell duct and the flow channel are in diffusive communication through the membrane and the porous membrane is adapted to prevent a cell in the cell duct from passing therethrough, while allowing a cell nutrient in the flow channel and a cell product in the cell duct to pass therethrough.

One or more of the following features may be included. The cell duct plate may include a material such as glass, fused silica, quartz, silicon, and/or organic polymers. The flow channel plate may include a material such as glass, fused silica, quartz, silicon, and/or organic polymers.

The porous membrane may include a material such as glass fiber, polycarbonate, polyethylene, polypropylene, polystyrene, polyimide, cellulose, nitrocellulose, cellulose esters, nylon, rayon, fluorocarbon, perfluorocarbon, polydimethylsiloxane, polyester, acrylics, acrylonitrile-butadiene-styrene, polyoxy-methylene, polyarylate, polyvinylchloride, PBT-Polyester, polybenzimidazone, acetal copolymers, polyimides, ethylene-chlorotrifluorethylene, PET polyesters, ethylene-tetrafluorethylene, fluorinated ethylene propylene, polyphenylene sulfide, polyethylene, polyurathanes, polyketones, polychloro-trifluoro-ethylene, polyvinylidene fluoride, polyethylene terephthalate polyesters, polypropylene oxides, polypropylene styrenes, polyether-ether ketones, polytetrafluorethylene, polyarylether sulfones, polyamide-imides, polyphenylene sulfides, polyarylates, polymethylpentene, polyketones, polysulfones, polyphenylene sulfides, PBT polyesters, and/or alloys of polymers.

The porous membrane may define a pore size having a diameter selected from a range of about 1 nanometer (nm) to about 100 micrometers (μm). The porous membrane may have a thickness less than about 200 μm; the thickness may be greater than about 5 μm. The porous membrane may include an interfacial layer disposed between the cell duct plate and the flow channel plate.

The microfluidic device may have a plurality of cell ducts in combination with a plurality of flow channels. A number of cell ducts may be equal to a number of flow channels. The cell ducts may be generally radially disposed about a common duct opening. At least two flow channels may not be in mixing fluidic communication with each other. At least two flow channels may be in mixing fluidic communication with each other. At least one of a cell duct and a flow channel may include a valve.

In another aspect, the invention features a microfluidic device for retaining a cell sample including a plurality of cells. The device includes a plate defining a common duct opening adapted to receive the cell sample and at least two cell ducts in fluidic communication with the duct opening, so that at least a portion of the cell sample can flow into a first cell duct and another portion of the cell sample can flow into a second cell duct.

One or more of the following features may be included. The plate may further define a manifold interdisposed between the duct opening and at least one cell duct. The device may include a pressure differential source adapted to induce the flow of at least one of the cell sample portions into at least one of the cell ducts.

In another aspect, the invention features a system for monitoring an activity of a cell. The system includes a microfluidic device including a cell duct plate defining at least one cell duct therein, a porous membrane having a first side bounding at least a portion of the cell duct; and a flow channel plate, defining at least one flow channel therein, at least a portion of the flow channel being bounded by a second side of the porous membrane. The porous membrane is adapted to prevent a cell in the cell duct from passing therethrough, while allowing a nutrient in the flow channel to pass therethrough and allowing a product of the cell to pass therethrough. The system also includes a pump adapted to induce flow of a nutrient media through the flow channel to support cell viability in the cell duct, a controller adapted to control flow in the microfluidic device, and a sensor adapted to detect at least one of the cell and the product of the cell.

One or more of the following features may be included. The sensor may include at least one of an electrochemical detector and a luminescence detector. The luminescence detector may include a fluorescent reagent, an excitation light source adapted to provide radiation having a first radiation wavelength range, and a detector adapted to measure an intensity of emitted light in a second radiation wavelength range, the second radiation wavelength range being different from the first radiation wavelength. The electrochemical detector may include an electrode adapted to measure at least one of pH and dissolved oxygen.

### BRIEF DESCRIPTION OF THE DRAWINGS

The following drawings are not necessarily to scale, emphasis instead being placed generally upon illustrating the principles of the invention. The foregoing and other features and advantages of the present invention, as well as the invention itself, will be more fully understood from the following description of exemplary and preferred embodiments, when read together with the accompanying drawings, in which:

FIG. 1a-1c are schematic top views of a microfluidic device, including with variations and portions thereof, in accordance with two embodiments of the invention;

FIG. 2 is a detailed view of a portion of the microfluidic device illustrated in FIG. 1;

FIG. 3 is a schematic cross-sectional view of a cell duct plate, a flow channel plate, and a membrane in accordance with one embodiment of the invention;

FIG. 4 is a schematic cross-sectional view of valve placement flow channels in accordance with an embodiment of the invention;

FIG. 5 is a schematic view of flow channels with valves, in accordance with an embodiment of the invention; and

FIG. 6 is a schematic view of an automated external pressure-controller device and microprocessor for use with the microfluidic device of FIG. 1 in accordance with one embodiment of the invention.

### DETAILED DESCRIPTION

Microfluidics:

It may be advantageous to use microfluidic devices to retain cells for sensitively monitoring cellular metabolism. Microfluidic devices have one or more channels of micrometer-size depth and width, generally between 10 μm and 900 μm in cross sectional diameter. The channels may be of widely varying length but generally are between 0.1 and 100 cm in length. Microfluidic devices may, therefore, contain small volumes defined by each channel, generally ranging from 100 picoliters to 100 microliters. Because of their small internal volume, microfluidic devices may have the following advantages:

a) Reagent consumption may be low;

b) Only a few cells may be required to create a measurable change in the extracellular concentration of cell metabolites;

c) The devices may be compact and easily stored;

d) The devices may be disposable and convenient to use; and

e) Changes in metabolic nutrients and products may be detected rapidly.

Low reagent consumption may be especially important when precious or rare reagents are used, particularly when the effect of purified cellular growth factors are being tested on cells. Low reagent consumption may also be important when drug candidates from chemical microlibraries are being tested on cells. A large synthetic effort may be required to create even small amounts of each reagent in such microlibraries because they may include a very large number of compounds, e.g., one-thousand to one-million compounds, or more.

Advantageously, the metabolic chamber may be small, so that a change in concentration of either nutrients consumed or cell products produced by the cells (e.g., metabolic products of the cells) may be large enough to be measured in a brief period of time. More precisely, there is a general requirement for a low ratio of analyzed extracellular volume V_{E} (of the metabolic chamber) to intracellular volume V_{I} of the cells contained in the chamber. The ratio V_{E}/V_{I} may be in the range of 10-1000. For mammalian cells in the range of 5-10 μm in diameter, the intracellular volume may be on the order of 0.05-0.5 picoliter. Thus the analyzed volume of a metabolic chamber may generally be between 0.5 picoliters and 1 nanoliter per cell. Employing 1000 cells therefore may require a device generally having an extracellular volume between 1 nanoliter and 1 microliter. Microfluidic devices having fluidic chambers, or compartments, of 1 microliter, or less, therefore are useful for such metabolic measurements. In embodiments where sensitivity is not the primary concern, the volume of the cell-retaining chamber may be larger, e.g., up to 1 microliter. In general, however, for adequate sensitivity, the cells may be contained in a small volume that is only a few times larger that the internal volume of the cells themselves.

The number of cells to be consumed during metabolic measurement in such microfluidic device may be low, for example 1 cell or a group of 2 or more cells, e.g., up to 100 cells, 1000 cells, or 10,000 cells.

Advantageous Properties of Microfluidic Devices:

The microfluidic channels used in microphysiometry may be formed from any substance having a surface compatible with biological cells. The channels (or at least the surface of the channels) may be made of, for example, glass, fused silica, quartz, or silicon. See, for example: Bousse, L., Cohen, C., Nikiforov, T., Chow, A., Kopf-Sill, A. R., Dubrow, R. and Parce, J. W. (2000) “Electrokinetically Controlled Microfluidic Analysis Systems.” Annu. Rev. Biophys. Biomol. Struct. 29, 155-181 and the references contained therein, hereby incorporated by reference.

Other materials that may be used for construction of microfluidic devices include organic polymers (i.e., plastics) such as methacrylates, polystyrene, polypropylene, polycarbonate, polyethylene, or the like. Soft polymeric materials such as organosilanes, including polydimethylsilane (PDMS) can be used to fabricate the microfluidic channels. The soft polymers alternatively may be polyacrylamide materials or mixed polymers containing co-polymerized organic or inorganic substances. A major advantage of soft polymers is that they may be deformable by the application of external pressure. Application of external pressure may result in creation of a closed valve. Because the soft polymer materials may be elastic, release of the pressure results in reopening of the valve. Flow in the channel may be restored provided that a gradient in pressure is created along the length of the channel. See, for example Thorsen, T., Maerkl, S. J. and Quake, S. R. (2002) Microfluidic Large-Scale Integration Science 298, 580-586 and the references contained therein, hereby incorporated by reference. Application of external pressure adjacent to a closed valve creates pressure that may be used to pump fluids. Alternatively, the pressure may be created by application of gas pressure, application of a vacuum (relative to ambient pressure) or by applying an electrical field along the channel and creating a pressure gradient by electroendosmosis. All of these processes are well known in the art of microfluidics.

Problems Encountered in Retaining Cells in a Microfluidic Device:

A major problem encountered when seeking to employ a low ratio of extracellular volume V_{E} to intracellular volume V_{I} (V_{E}/V_{I}) needed for sensitive metabolic measurements is that the cells rapidly consume their nutrients, such as oxygen, glucose, amino acids, etc. These cellular nutrients often are essential for continued cellular metabolism. Also, the cells excrete their metabolic products, such as protons, CO₂, ammonia, lactic acid, etc., into the very small extracellular volume around the cells. Since such products of cellular metabolism at high concentration are often toxic to the cells, excretion into the small volume can create serious toxic effects on the cells. In order to circumvent these problems, it is generally useful to retain the cells in a flowing fluid stream. The flowing stream may generally have a linear velocity of between 0.1 and 1000 cell lengths per second and serve to maintain a supply of nutrients to the cells and to remove toxic waste products from their vicinity.

The fluid stream may have an intermittent flow, where the flow is turned off for a brief period of time, e.g., for 1-100 seconds. During the period that the flow is stopped, the rate of change in the concentration of cell nutrients (or alternatively metabolic products) may be measured. After a predetermined period of time, the flow may then be resumed to bring fresh nutrients to the cells. This process may then be repeated. The cycles may be repeated any number of times up to 10, 100, 1000, or more times. For brief analysis, the cycle may be repeated only once. For longer-term analysis, the cells may be analyzed for a multiplicity of cycles repeated for more than 1 hour, up to 24 hours, 36 hours, or 48 hours, or more. The rate of change in a measured metabolic rate may be determined by comparison of the measured metabolic rates between any two cycles. Sequential comparison of successive cycles allows a time course of metabolic change to be determined. The temporal resolution of the measurement is the interval of time between cycles.

Biological cells to be retained for metabolic analysis may include any biological cell, which may be plant or animal cells. The cells may be larger than 2 μm in diameter but less than 100 μm in diameter. The cells may be eukaryotic mammalian cells. The cells may be samples taken directly from plant or animal tissues (primary cells) or may be grown in tissue culture. The cells may be altered genetically (for example, enriched in cellular receptors for cell-affecting agents such as cytokines, endorphins, hormones, cell nutrients, cell toxins and the like). Also, the receptors may effect a cellular function. Such cellular receptors also may bind agonists or antagonists (respectively, that stimulate or inhibit stimulation of a receptor's cellular function). Thus such cells may be especially useful for screening potential drug candidates for use in modifying receptor activity in cells.

Measurement of Cell Metabolism by the Rate of Consumption (or Production) of Metabolic Substrates (or Products):

Physiological measurements to be carried in the microfluidic device may include measuring the rate of one or more of the following:

a) consumption of oxygen,

b) production of protons (e.g., measurement of intracellular or extracellular pH or their rate of change relative to the available buffering capacity),

c) production of carbon dioxide,

d) production of lactic acid, or

e) production of cell-signaling molecules including:

i. intracellular cell-signaling components (e.g., cyclic AMP or GMP, cytosolic Ca⁺² ion concentrations, inositol-phosphates, etc.) and

ii. extracellular cell signaling components or hormones (e.g., insulin, growth factors, interleukins, cytokines, etc.)

Analytical Sensors for Cell Metabolism:

In general, sensors of cellular metabolism that may be incorporated into a cell-retaining device include electrochemical detectors (such as glass or metal electrodes for the measurement of pH, redox potential, oxygen, or specific ion electrodes) are well known in the prior art. The preferred mode of metabolic measurement, however, may be to monitor luminescence related to cellular metabolism. Luminescence monitoring has the advantage that no electrode structures or conductive leads in the cell-retaining device are necessary. It is only necessary that the device be transparent to light. The emission of such luminescent light may be stimulated by excitation irradiation, as is well known to occur in fluorescence and phosphorescence.

A luminescent probe maybe introduced into the cell-retaining device and maintained in diffusive communication with metabolizing cells in order to monitor their metabolic properties. The luminescent sensors include fluorescent probes. Such fluorescent probes or labels may be introduced into cells, or their communicating extracellular environment, for monitoring a wide variety of important cellular responses including transmembrane potential or intracellular calcium. See for example, the Handbook of Fluorescent Probes published by Molecular Probes, Inc., Eugene, Oreg., USA, and the references contained therein, hereby incorporated by reference. Also, fluorescent probes for sodium, potassium, calcium or chloride channel conductances are of great interest. Similarly, fluorescent ligands that specifically bind to selected biological moieties, including proteins, lipids, or carbohydrate moieties, may be used to monitor the presence of such moieties. Especially useful for specifically labeling specific cellular components include fluorescently-labeled specific antibodies, or alternatively, fluorescently-labeled lectins which are proteins which bind specific carbohydrate moieties. Another extremely useful group of fluorescent labels are those used to monitor the presence of single-stranded or double-stranded DNA, RNA or other oligonucleotides. Also, specific sequences of DNA, RNA or other oligonucleotides may be detected or quantified by using labeled, complementary, DNA, RNA or other oligonucleotides, as is well known in the prior art.

Still other types of metabolic sensors may be used to monitor chemiluminescence from cells or cell metabolites. For example, intracellular calcium levels may be determined by introducing aequorin into cells. Aequorin, a protein from the jellyfish Aequoria Victoria, catalyses the oxidation of coelenterazine by oxygen, but only in the presence of Ca⁺⁺ ions. Aequorin may be introduced into cells by incorporating cDNA encoding for the aequorin messenger RNA into the cellular DNA within targeted domains of selected intracellular proteins, as is well known in the art of genetic engineering. Similarly, the genes for expression of enzymes necessary for coelenterazine production in the cellular cytosol may be incorporated into the cells. When intracellular calcium concentration rise in such cells, chemiluminescent light from the aequorin-catalyzed oxidation of coelenterizine will be emitted and can be detected by a photodetector device. See, for example, Pouli, A. E., Karagenc, N., Arden, S., Bright, N., Schofield, G. S., Hutton, J. C. & Rutter, G. A. (1998) “A phogrin-aequorin chimaera to image Ca²⁺ in the vicinity of secretory granules.” Biochem. J., 330, 1399-1404, hereby incorporated by reference.

Another well-known example of a chemiluminescent indicator of cellular metabolism is to use the luciferase enzyme obtained from fireflies (or the cloned gene from luciferase introduced in other cells) together with oxygen and luciferin to monitor the concentration of ATP released from biological cells. Other metabolites, e.g., pyrophosphate, AMP, ADP, etc. may be monitored also by virtue of coupling to this luciferase reaction by including the enzymes or substrates required to covert such substrates into ATP. Still another chemiluminescent technique is to monitor the presence of flavin mononucleotides (FMN or FMNH), or biologically interconvertable metabolites such as FAD, FADH, etc. by using bacterial luciferase instead of firefly luciferase.

Photodetector Devices:

When detecting fluorescent or chemiluminescent light emission, a photodetector device, such as a photomultiplier tube, a photodiode, a charge-coupled device (CCD), or a complementary metal-oxide semiconductor (CMOS) device may be placed in close proximity to the cells. Alternatively, an optical lens or mirror may be used to collect the light and focus it onto the light-detector, as is well known in the prior art. The photodetector may be an array of photodetectors allowing spatial characteristics of the light to be determined. Such characteristics include the spatial distribution of light emission from within a sample, i.e., the image of the light intensity emitted from the cells (or from an array of samples). Alternatively, after passing the emitted light through a wavelength-dispersion device, such as an optical prism or grating the wavelength distribution (i.e., the wavelength spectrum) of the emitted light can be deduced, as is well known in the prior art.

Light Sources for Excitation of Fluorescence:

Fluorescence of samples may be stimulated by an excitation light beam of radiation. The excitation light may be from a white light source, such as incandescent filaments or from plasma emission tubes that may emit either continuously or as light flashes (e.g., a Xenon flash lamp). The light for excitation may be passed through a monchromator (e.g., optical filters or alternatively a prism or grating monochromator including an optical exit slit) prior to impinging on a fluorescent sample. The monochromator thereby serves to remove unwanted wavelengths of light from the excitation radiation allowing sensitive detection of emitted light, as is well known in the prior art. In some embodiments, monochromatic light sources such as lasers or light-emitting diodes may be used for excitation of fluorescence. Such sources of light can be very intense and highly monochromatic, thereby providing for greater sensitivity in detection of fluorescent molecules.

Use of Fluorescent Indicator Particles:

Fluorescent or luminescent species conveniently may be retained within particles for specifically monitoring metabolic species in the extracellular environment near biological cells including oxygen and glucose. See, for example, Lahdesmaki, I, Scampavia, L. D., Beeson, C. Ruzicka, J. (1999) “Detection of oxygen consumption of cultured adherent cells by bead injection spectroscopy.” Analytical Chemistry, 71, 5248-5252, and Wiley, C and Beeson, C. (2002) “Continuous measurement of glucose utilization in heart myoblasts.” Analytical Biochemistry 304, 139-146; both references hereby incorporated by reference. In the present invention, such particles may be used in microfluidic devices, together with cells, to sensitively measure the changes in concentration of metabolic products caused by cells retained in diffusive communication with the particles.

Cell-Retaining Fluidic Devices:

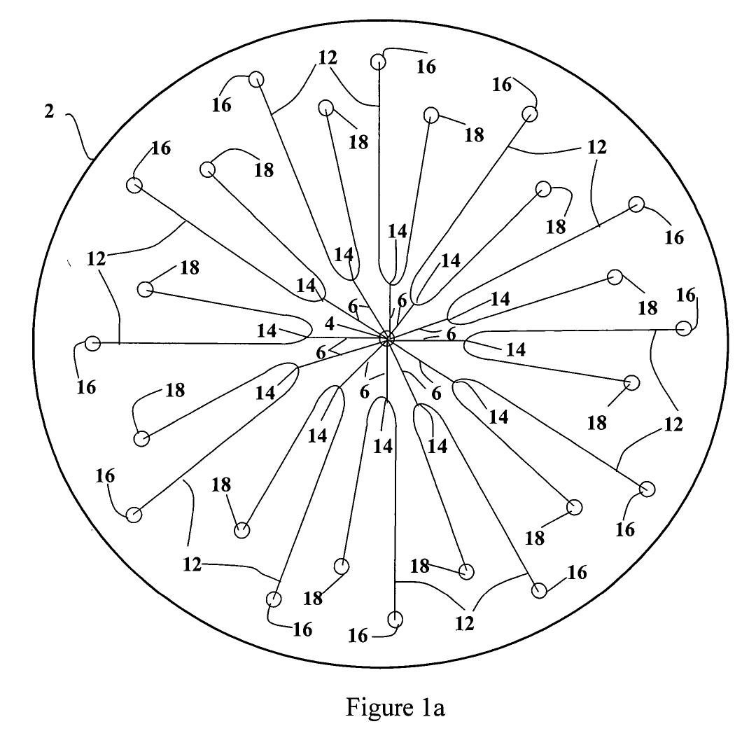

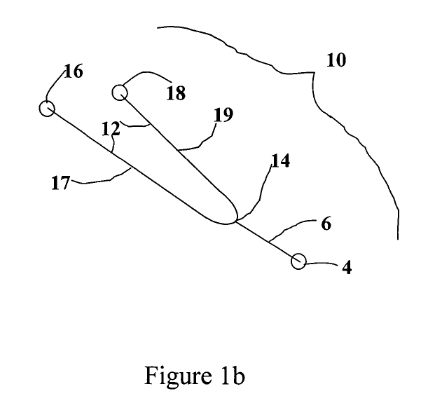

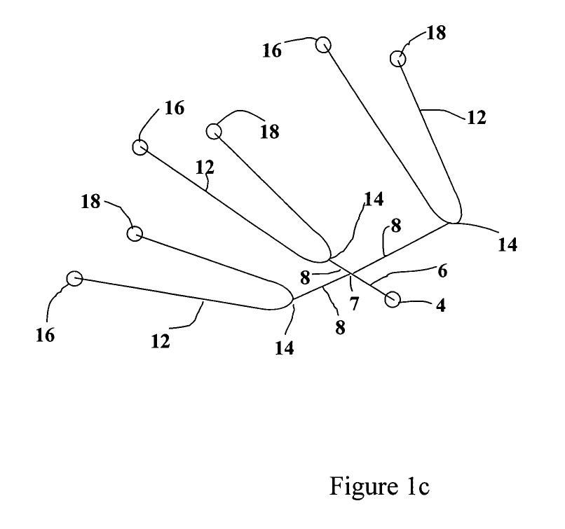

Referring to FIG. 1a, a microfluidic device 2 for retaining and studying the physiology of cells may have at least one external duct opening 4 forming a “cell well” to admit cells into cell ducts 6. A single external opening 4 advantageously may be placed in the center of a generally circular microfluidic device. Two, or more, cell ducts 6 may intersect with one common duct opening 4. The cell ducts may be generally radially disposed about the common duct opening.

With this embodiment, the number of cell wells required for a large number of cell ducts and also the number of addition steps in providing cells to the cell wells may be reduced and the task thus simplified. Also, the number of cells required to fill the cell wells prior to operation of the device may also be reduced.

The microfluidic devices for retaining cells may also have one or more nutrient media-supplying flow channels 12. The cell ducts 6 and channels 12 intersect at predetermined intersection regions 14. The intersection region may be quite small, such as in an embodiment where two narrow microfluidic channels intersect at a high angle of approach that is, e.g., close to 90 degrees. Alternatively, the region of intersection 14 may be extensive, e.g., extending substantially along the entire length of either the cell ducts 6 or nutrient-supplying channels 12 by closely aligning the ducts and channels to be contiguous along a substantial fraction (e.g., >50%) of either length or of both. In an embodiment, each individual, unique cell duct 6 may intersect with one individual, unique flow channel, so that there is a one-to-one relationship between cell ducts and flow channels, i.e., a number of cell ducts is equal to a number of flow channels. More complex fluidic arrangements, however, may also be used where there are two or more cell ducts intersecting with one flow channel, or alternatively where there are two or more flow channels intersecting with one cell duct.

Referring to FIG. 1b, a single operative cell-monitoring unit 10 includes one cell duct 6 and one channel 12. Each microfluidic device 2 may have a multiplicity of two or more operative cell-monitoring units 10, each of which includes at least one external duct opening 4, connected to one or more cell ducts 6, each having an intersection region 14, at porous membrane 20. The porous membrane may be in diffusive communication with flow channel 12, having a first opening 16 for introduction of fluid, and a second opening 18 for flow exit. The number of operative units may advantageously be large, e.g., between 10 and 100. As shown in FIG. 1a, the number of operative units may be 12, but with suitable microfluidic engineering can be much greater, for example up to 1000 or more.

The flow channels 12, used to supply nutrient media fluid, may have at least two external openings 16, 18. A first flow channel opening 16 may be used to introduce fluid media into the flow channels 12 where it can flow to the cells present at duct intersection points 14. After flowing past the intersection regions 14, the fluid media may exit the flow channel through a second flow channel opening 18. The first channel opening 16 may generally be referred to as an “upstream” flow channel opening and the second channel opening 18 may generally be referred to as a “downstream” flow channel opening. Similarly, a portion of each flow channel 12 that is between the upstream opening 16 and intersection region 14 may generally be referred to as an “upstream” portion of flow channel 12. Further, a portion 17, of each flow channel 12 that is between intersection region 14 and the downstream opening 18 may generally be referred to as a “downstream” portion 19, of flow channel 12.

Referring to FIG. 1c, alternatively, the ducts 6 may have one or more duct bifurcations 7 into subsidiary cell ducts 8, thereby forming a manifold region 9, interdisposed between duct opening 4 and cell ducts 8. In this embodiment, the bifurcations are arranged so that cells may be added in one well, such as external duct opening 4, and by flowing through the one or more bifurcations may enter two, or more, subsidiary cell ducts 8. Thus, the number of cell ducts intersecting at the external opening 4 may be reduced to a number less than the total number of functional operative cell-monitoring units, each having at least one cell duct.

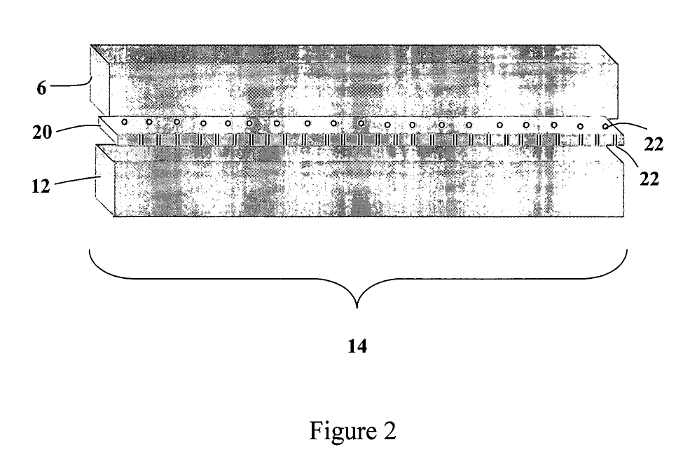

Referring to FIG. 2, one of the intersection regions 14 is shown in greater detail. The ducts 6 and channels 12 are separated by a cell-retaining porous membrane 20 with pores 22. The pores are sufficiently narrow in diameter to prevent the retained biological cells from passing through the membrane and, e.g., entering the flow channels 12. The pores 22, however, are large enough to allow nutrients from the media to reach the cells by passing through the membrane (either by diffusion or by fluid flow) and to allow gases such as oxygen and carbon dioxide to pass freely. Also the pores 22 allow cell products, such as metabolic products from the cells to reach the flow channels 12 by passing through the membrane 20 (either by diffusion or by fluid flow). The membrane, therefore, provides diffusive communication between its two sides. Depending on the nature of the cells employed and the nature of the cell nutrients and cell products, e.g., metabolites, the pores sizes may include diameters as large as 100 μm or as small as 1 μm. In general the pore size will be less than 10% of the smaller of either the depth or width of the cell ducts 6 but greater than 1 nm.

The cell-retaining membrane 20 may be formed from any suitable material including glass fiber, polycarbonate, cellulose, nitrocellulose, nylon, rayon, polyester, e.g., Dacron®, or the like. The membrane 20 may be tightly or loosely woven, or may be a track-etched solid membrane, such as a Nucleopore membrane. The membrane may be a single layer or may be formed from multiple layers as is well known in the art of membrane manufacture. Numerous manufacturers of suitable membranes exist including, Millipore, Nucleopore, Pall, Gelman and Whatman. Advantageously, the membranes are chosen to be relatively thin, generally between 1 μm and 200 μm, e.g., between 10 μm and 50 μm in thickness, so that the rate of nutrient transport across the membranes may be rapid.

Additional materials that may be used from the membrane include acrylics, e.g., LUCITE® or Plexiglas; acrylonitrile-butadiene-styrene (ABS); polyoxy-methylene (Acetal); polyarylate (ARDEL®); polyvinylchloride (PVC); PBT-Polyester (CELANEX®); polybenzimidazone (Celazole®); the acetal copolymers Celcon, or Delrin®; polyimides, e.g., Duratron® or Kapton®; ethylene-chlorotrifluorethylene, e.g. Halar®; PET polyesters, e.g. Ertalyte®; ethylene-tetrafluorethylene, e.g. Tefzel®; fluorinated ethylene propylene (FEP); polyphenylene sulfide; polyethylene; polyurathanes, e.g., Isoplast®; polyketones, e.g. Kadel®; polychloro-trifluoro-ethylene (Kel-F®); polyvinylidene fluoride (PVDF); polyethylene terephthalate polyesters, e.g,—Mylar®; polypropylene oxides and styrenes, e.g. Noryl®; polyether-ether ketones, e.g. PEEK™; polytetrafluorethylene (Teflon®); polyarylether sulfones, e.g. Radel®; polyamide-imides, e.g. Torlon®; polyphenylene sulfides, e.g. Techtron®; polyarylates, e.g. Ardel®; polymethylpentene (TPX®); polyketones, e.g. Kadel®; polysulfones, e.g. Udel®; polyphenylene sulfides, e.g. Ryton®; PBT polyesters, e.g. Valox®; membranes formed from alloys of polymers, e.g. Xenoy®; or laminates of two or more polymer membranes.

Highly impermeable polymers, e.g., Mylar® advantageously may be used because of their high burst strength, thereby allowing very thin membranes to be used. In such embodiments, however, the impermeable polymer may first be perforated, for example, mechanically, by electrical breakdown, or by optical (e.g., laser) means in order to increase the permeability of these membranes to oxygen, CO₂, or other cell nutrients and products. The size of the perforation may be selected to optimally retain the cells and to restrict convection through the membrane as desired.

The size of the pores of the porous membrane may be selected to be relatively large, where mixing communication between the cell duct and flow channels is desired. In this case, substantial flow and convection of fluid across the porous membrane occurs. For mixing communication, the pore size may be between 1 μm and 50 μm in diameter or larger. In contrast, where mixing communication is not desired, but instead the communication is selected to be solely by diffusive communication, the pore size may be smaller, e.g., between 10 nm and 1 μm in diameter.

Advantageously, the cell ducts may be filled by displacing fluid in the cell ducts through the membrane 20. Thereby the cell ducts 6 may have only a single duct opening 4 for introduction of the cells, as shown in FIG. 1a. In this embodiment, the membrane 20 may be selected to have surface properties that allow the membrane to wet easily with the fluid medium suspending the cells in the cell duct. For example, if the medium is an aqueous medium, then the membrane may be hydrophilic, allowing the aqueous medium to pass through the membrane, but retaining the cells. This latter mode of operation stacks the cells against the membrane 20 at the intersection regions 14, thereby concentrating the cells in these regions. Alternatively, in a second mode of operation, the membrane may be selected from a group of polymers, e.g., Teflon®, polyethylene, or any other hydrophobic polymer that has a high permeability to air. In this mode of operation, the cell ducts may be filled by placing an aqueous suspension of cells in the cell duct openings 4 and the cell ducts fill by

a) capillary forces (e.g. by wetting of hydrophilic surfaces of the cell ducts).

b) application of a positive pressure to the cell duct openings, or

c) application of a negative pressure, i.e., a vacuum, to the flow channel opening 16 and 18, or

d) any combination of a) through c).

Filling by capillary action may be particularly convenient and may be employed when the cell duct surfaces wet with the cell suspension fluid and when the membrane 20 is selected to be hydrophobic, but to also have a high permeability to the gas (e.g. air) present in the cell ducts prior to filling. To improve the wetting properties of the surfaces of the cell ducts, the cell duct surfaces may be treated with an oxygen plasma, as is well known to those skilled in the art, to improve the wetting properties of polymer surfaces. This treatment may be done prior to application of membrane 20 so that the membrane remains hydrophobic, so that the cell-suspending medium is retained with the cells in cell ducts 12.

Retention of the cell-suspending medium in the cell ducts 12 may have the advantage of maintaining a constant concentration of cells in the cell-suspending medium during the duct-filling procedure. Also the cells may not have any propensity to clog the membrane 20 during the filling procedure. This mode of filling may particularly advantageous when the region of intersection 14 between the cell ducts and the flow channels is extensive, e.g., along a substantial (e.g. >10%) of the length of either the cell ducts 6 or nutrient-supplying flow channels 12. Such a construct may be fabricated by closely aligning the ducts and channels to be contiguous along a substantial fraction of either. Such methods of alignment of two parallel channels are well known to those skilled in the art of microfluidic channel fabrication. In general the alignment can be done by optical means, either in an automated mode or by manual observation.

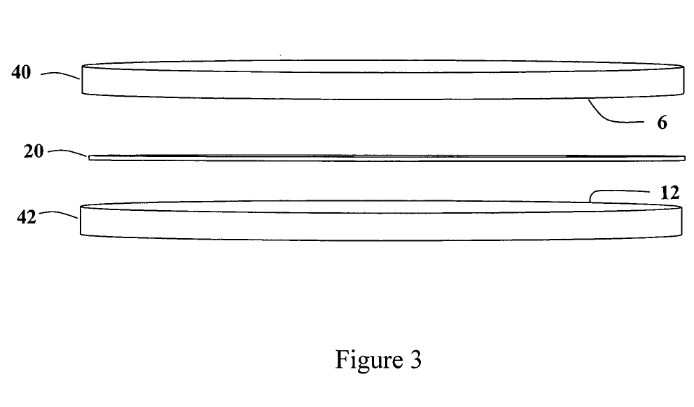

Referring to FIG. 3, the ducts 6 and the media-supplying flow channels 12 advantageously may be fabricated in two separate plates, a cell duct plate 40 and a flow channel plate 42. Also advantageously, the cell-retaining membrane 20 may be positioned as an interfacial layer between the cell duct plate 40 and flow channel plate 42. Either or both of the cell duct plate and the flow channel plate may be formed from glass, fused silica, quartz, silicon, and/or organic polymers. During manufacture, the two plates and the cell-retaining membrane may be fused together with suitable techniques for fusion of the plate materials. Suitable adhesive materials include RTV silicone adhesives, such as those recommended by Dow-Corning for similar applications. Alternatively, the plate materials may be heated to promote plate fusion and adhesion of the two plates to the porous membrane material. For glass or ceramic plates, anodic bonding techniques, as well known to those skilled in the art, may be used to assist the bonding process.

Structures and Methods for Regulation of Flow:

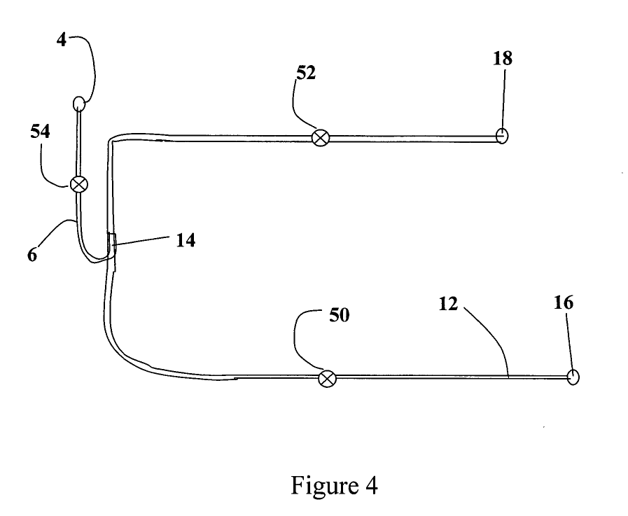

Referring to FIG. 4, valve structures advantageously may be placed in the flow channels 12. In each channel a first valve 50 may be placed between the first external channel opening 16 and duct intersection point 14. Similarly, in each channel a second valve 52 may be placed between duct intersection point 14 and the second external channel opening 18. Optionally, in each cell duct 6 a third valve 54 may be placed between flow channel intersection points 14 and a first-encountered duct bifurcation 32, if any. If no duct bifurcation is present, the third valve may be placed anywhere in the cell ducts 6 between flow channel intersection points 14 and duct external opening 4.

Valves 50 and 52 in flow channels 12 help ensure that the flow in channels 12 can be turned on and off in a reproducible manner during the monitoring of cellular metabolism. An optional valve 54 in cell duct 6 may be used to ensure that cells trapped at intersection points 14 are locked into place and cannot flow backwards in the cell ducts during metabolic measurements.

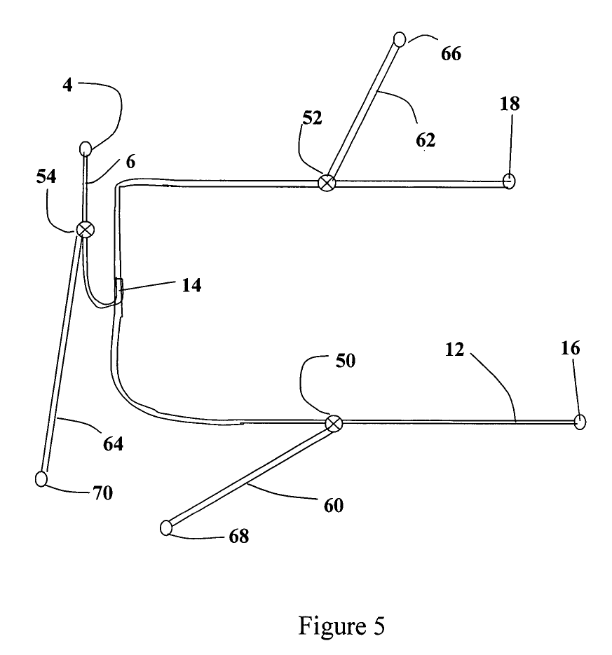

Any number of methods may be used to manufacture valve structures 50, 52, and 54, as is well known to those skilled in the art. For example, the valves may be made of the silicone elastomer polydimethylsiloxane (PDMS) and operated by applying external pressure to open and close the valves as described by Unger, M. A., Chou, H-P, Thorsen, T., Scherer, A, and Quake, S. R. (2000) Science 288, 113, hereby incorporated by reference. Referring to FIG. 5, alternatively, individual valve structures may be created by incorporating valve channels 60, 62, and 64. The valve channels have external openings 66, 68, and 70 to which fluid pressure (i.e., a gas or liquid) may be applied to actuate the valves. The valve channels may terminate, respectively, at valve structures 50, 52, and 54. Each valve may include a valve chamber having a flexible solid plug, made of, for example, polyacrylamide. The fluid pressure causes the solid plug to slide forward within the valve chamber and against a valve seat within a flow channel 12. This action effectively turns off the flow within flow channel 12. See, for example, Hasselbrink E. F. Jr., Shepodd T. J., Rehm J. (2002) “High-pressure microfluidic control in lab-on-a-chip devices using mobile polymer monoliths.” Anal Chem. 74, 4913-8, hereby incorporated by reference. Optionally, this type of valve also may be used in cell ducts 6.

Other means for constructing valves in microfluidic structures may be used as well. For example, gas bubbles may be injected or formed electrochemically to create microfluidic valves. See for example, Hua S. Z., Sachs F., Yang D. X., Chopra H. D. (2002) “Microfluidic actuation using electrochemically generated bubbles.” Anal Chem. 74, 6392-6, incorporated hereby by reference. Another method of effecting a microfluidic valve is by freezing a liquid in a microfluidic channel to become a solid. Freezing may be caused by cryogenic cooling of a small region of the channel. Formation of the solid results in a closed valve. Subsequently, thawing the solid results in opening of the valve. The freezing and thawing cycle may be repeated any number of times to open and close the valve in a cyclic fashion. Still other microfluidic valve means can be found in: Beebe D. J., Mensing G. A., Walker G. M. (2002) “Physics and applications of microfluidics in biology.” Annu. Rev. Biomed. Eng., 4, 261-86 and in: Beebe D. J., Moore J. S., Bauer J. M., Yu Q., Liu R. H., Devadoss, C., Jo B. H. (2000) “Functional hydrogel structures for autonomous flow control inside microfluidic channels. Nature 404, 588-90, both references hereby incorporated by reference.

Flow Control and User Interface:

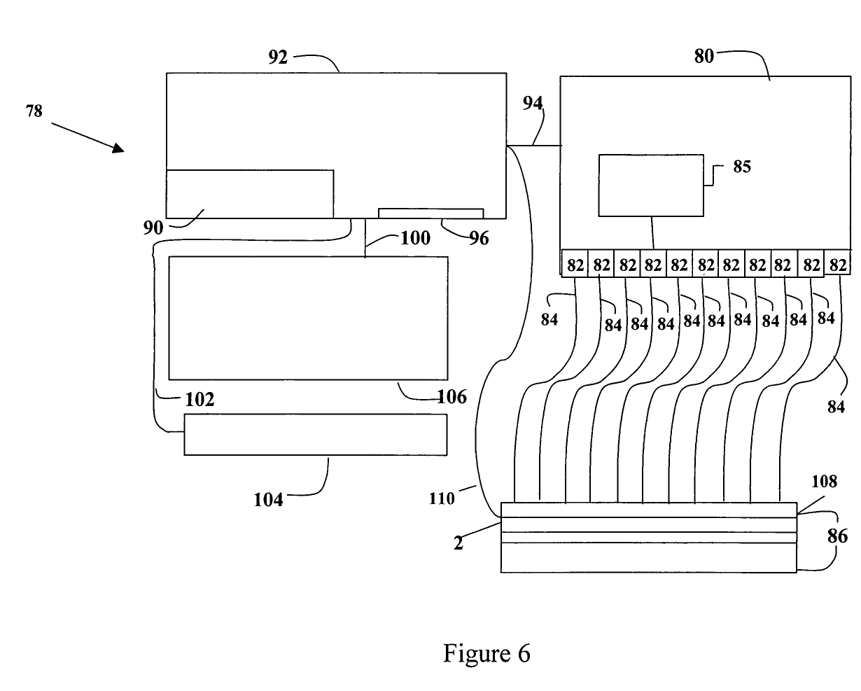

Referring to FIG. 6, a system 78 for monitoring an activity of a cell may include an automated external pressure-controller device 80 having a multiplicity of pressure controllers 82, each with a tubing connector 84. The pressure-controller device 80 may include a pump 85, adapted to control flow in microfluidic device 2 in conjunction with the pressure controllers 82. The pump may be adapted to induce flow of a nutrient media through the flow channel to support cell viability in the cell duct. Each tubing connector interface, in a sealing manner, to the external openings 4, 16, 18, to the cell ducts 6, and to the flow channels 12 of the microfluidic device 2. The sealing connection may be facilitated by a cartridge 86 that may both retain the microfluidic device 2 and apply pressure to suitable O-rings that form a sealing connection between the cartridge and the external openings 4, 16, 18 to the microfluidic device. Other suitable O-rings within the cartridge 86 may also form a seal between the cartridge and tubing connectors 84. Similarly, additional pressure controllers 82 and tubing connectors 84 are used to control valves 50, 52, 54 by interfacing (within cartridge 86) in a sealing manner with the external openings 66, 68, 70 that connect the valve channels 60, 62, 64, to the tubing connectors 84.

The external pressure controller device 80 may be controlled by a microprocessor 90 within a computer 92, via an interface cable 94 leading from the computer to the pressure controller device 80. The computer 92 may have a graphical user interface program 96 for control of the microfluidic device by the user. The computer 92 may be connected via cable connectors 100 and 102 to a keyboard 104 and to a computer monitor 106 for user interaction, data entry and visualization of device operations and data acquisition. The graphical user interface program 96, together with the other automated controls, greatly simplify operation of the microfluidic device. The automated pressure control may be used to establish desired pressure and flow rates in each flow channel 12 and cell duct 6. The automated pressure control optionally also may be used to establish desired pressure in valve channels 60, 62, 64, when control valves 50, 52, 54 are used. The microprocessor 90, computer 92, graphical user interface program 96, and computer monitor 106 also may be used to control and/or receive data from a sensor 108, as well as to process, store, and display selected data to the user. Sensor 108 may be in electrical and/or optical communication with computer 92 by a connector 110, e.g., an electrical cable, an optical cable, and/or a wireless connection. The sensor 108 may be an electrochemical detector, such as an electrode adapted to measure at least one of pH and dissolved oxygen. Alternatively, sensor 108 may be a luminescence detector, having a fluorescent reagent, an excitation light source adapted to provide radiation having a first radiation wavelength range, and a detector, e.g., a photodetector, adapted to measure an intensity of emitted light in a second radiation wavelength range, the second radiation wavelength range being different from the first radiation wavelength.

Control of Temperature:

Temperature has a marked effect on cellular metabolism. Therefore, temperature control may be important for any device used for retaining and studying the physiology of cells. Temperature of microfluidic device 2 may be controlled simply by placing the device in a temperature-controlled chamber. In a preferred mode, a temperature control is incorporated into cartridge 86 so as to provide for heating of the cartridge to selected temperatures between room temperature, i.e., about 23 degrees centigrade, and about 42 degrees centigrade. Typically, a temperature of the cartridge may be controlled to be about 37 degrees centigrade. Temperature control elements may include heating elements, temperature-measuring elements, e.g. thermistors or thermocouples, and controlling electronics (together with electrical leads and contacts) so that a stable temperature can be achieved. For example, a microprocessor may be used to coordinate interaction of the temperature control elements, employing for example a proportional/integral/differential (PID) algorithm to effect stable temperature regulation, as is well known in the art of temperature control.

Control of Composition and Concentration of Dissolved Gases:

The dissolved gas composition in nutritional media supporting cell metabolism may be important to the metabolic state of cells. For example, dissolved oxygen may be required to support cell metabolism, depending on the type of cells employed. Similarly, the concentration of dissolved carbon dioxide may be important for biological reactions requiring CO₂. Also, the concentration of CO₂ may be important for the regulation of media pH.

Also, excessive dissolved gases, even if the gas is relatively inert, such as nitrogen gas, can cause bubbles to form in fluidic channels. Such bubbles may interrupt fluid flow within the channels and thus compromise operation of cell-retaining fluidic devices. Heating of the microfluidic device may exacerbate bubble formation. To prevent bubble formation, media introduced into the fluidic device may be degassed prior to introduction. Degassing of media may be performed by any number of methods including applying a vacuum, heating, or any combination thereof.

In a preferred mode of operation, external pressure-controller device 80 may have attached sources of gases, such as oxygen, carbon dioxide, helium, or argon. The sources of gases may be in the form of compressed gas, cryogenically liquefied gases, or chemically generated gases. The gas composition may be adjusted to form a selected mixture of different gases. The gas mixture may then be applied as pressure to the fluidic channels. To avoid nitrogen bubbles from forming in the channels, other gases may be substituted for nitrogen. For example, inert gases, such as helium or argon may be used in the mixture. Advantageously, additional oxygen may be incorporated into the mixture when additional oxygen is needed to support cellular metabolism, as is predetermined and selected by users of the fluidic devices.

Operation of a Cell-Retaining Fluidic Device:

Cell-Loading:

In operation, selected biological cells (e.g. CHO-1 cells) in nutrient medium, (e.g. RPMI medium containing 5% fetal calf serum) may be introduced into a duct opening 4 together with 10% (by volume) of cell biology-grade, 20 μm diameter agarose particles. In a preferred mode of cell addition, a density of the cells may be determined by placing the cells in a density gradient and measuring the buoyant density of cells. The nutrient medium is then mixed with an isotonic solution of high density medium, such as Ficoll-Hypaque™ or Optiprep™, made up according to the manufacturers' recommendations so that the cell suspending medium is both isotonic and isopycnic. Making of such medium is well known to those skilled in the art of cell manipulation and cell fractionation. In such a way, the cells remain substantially in neutral suspension in the isopycnic solution. Thereby, the cells, when added to the cell ducts, remain at substantially constant cell concentration because the cells neither settle to the bottom nor float to the top of the suspending medium.

Applying a pressure to the duct opening 4 or vacuum to external channel openings 16, 18 may cause the cell suspending medium to flow. At least a portion of the cell sample can flow into a first cell duct 6 and another portion of the cell sample can flow into a second cell duct.

Gentle vacuum [about −1.0 pound per square inch (psi)] creating a pressure differential may be applied to external channel openings 16, 18. Optionally, positive pressure may instead be applied to duct openings 4 to cause cells to move to intersection regions 14. The vacuum (or positive pressure) may be discontinued when the nutrient flow channels have filled with nutrient medium. Subsequently, the remaining cell suspension in the duct opening 4 may be removed and replaced with nutrient medium. The vacuum to external channel openings 16 and 18 may then be reapplied, or alternatively, positive pressure may be applied to duct openings 4 so that the cells present within the cell ducts 6 may be carried to intersection points 14 where they may be retained by membrane 20. Advantageously, automated external pressure-controller device 80 may control these operations so that they may be carried out quickly and efficiently so that the cells are not left without flow for more than 1 minute. Application of substantially equal vacuum (negative pressure) to each of the external channel openings 16, 18 or application of positive pressure to duct openings 4 results in substantially uniform partitioning of a substantially identical concentration of cells in each cell duct. Thereby, a substantially equal number of cells is present at each intersection region 14 of the cell ducts in diffusive communication with the flow channels 12, where the rate of release of metabolic products from the cells is measured.

Monitoring Cellular Metabolism during Intermittent Flow:

After the cells are loaded into the intersection regions 14, the rates of cell metabolism may be monitored by turning on a light excitation source (for fluorescence measurements) to irradiate the cell intersection regions 14 and by collecting fluorescent (or luminescent) light emitted from the intersection regions 14. The excitation light may be directed to the intersection points 14 either from the cell duct side of the porous membrane 20 or from the flow channel side of the porous membrane 20. Generally, excitation will be from the cell duct side if the indicator fluorophores are present either within the cells, or within particles trapped together with the cells. Alternatively, fluorescence excitation generally will be from the flow channel side if the metabolic indicator fluorophores are present either as soluble fluorescent probes within the flow channels 12 or are attached to particles present within the flow channels 12.

The flow in flow channels 12 may then be cycled between the flowing conditions by creating a pressure gradient along channels 12. The pressure gradient may be created by applying either pressure (or vacuum) to external channel openings 16 or 18. Generally the pressure (negative or positive) will be small, e.g., between 0.1 psi and 3.0 psi, to reduce gas bubble formation within the channels. The flow may be turned on for a predetermined period. The duration of the “on period” may be selected to match the time resolution desired for the measurements. Generally, for greater resolution of time, the flow may be turned on only briefly, from between 1 second and 1 minute. When less resolution is needed, however, the flow may be maintained for longer periods of time, for example from 1 hour to 24 hours. The rate of metabolic change may be measured during the period of time that the flow is turned off. The “off period” may be selected generally to be a short interval, also between 1 second and 1 minute, for high time resolution. In any case, the “off period” is preferably long enough so that measured metabolic rates of the cells are high enough to be measured with precision. The precision in measuring metabolic rates during each “off period” generally will be between 0.1% and 5% coefficient of variation.

### EXAMPLE 1

### Monitoring of Extracellular pH in Microfluidic Cell-Retaining Devices

Various fluorescent indicators are described in the “Handbook of Fluorescent Probes” available from Molecular Probes, Inc. (Eugene Oreg.) and hereby incorporated by reference. For monitoring extracellular pH, pH-sensing dyes are available from Molecular Probes and may be incorporated into the fluid medium present in the flow channels 12 to monitor extracellular pH. For example, the carboxy SNARF-1 dye (catalog, No. C-1270) has a pKa of about 7.5 at room temperature and 7.3-7.4 at 37° C. Thus, carboxy SNARF-1 is useful for measuring pH changes between pH 7 and 8. Like fluorescein and 2′,7′-bis-(2-carboxyethyl)-5-(and-6)-carboxyfluorescein (BCECF), the absorption spectrum of the carboxy SNARF-1 pH indicator undergoes a shift to longer wavelengths upon deprotonation of its phenolic substituent. Any of these fluorescein-based pH indicators, or other non-fluorescein-based luminescent pH indicators, may be used to monitor pH in the instant invention. In contrast to the fluorescein-based indicators, however, carboxy SNARF-1 also exhibits a significant pH-dependent emission shift from yellow-orange to deep red fluorescence under acidic and basic conditions, respectively. This pH dependence advantageously allows the ratio of the fluorescence intensities from the dye at two emission wavelengths—typically 580 nm and 640 nm—to be used for quantitative determinations of pH. For practical purposes, as recommended by Molecular Probes, it is often desirable to bias the detection of carboxy SNARF-1 fluorescence towards the less fluorescent acidic form by using an excitation wavelength between 488 nm and the excitation isosbestic point at ˜530 nm, yielding balanced signals for the two emission-ratio components. When excited at 488 nm, carboxy SNARF-1 exhibits an emission isosbestic point of ˜610 nm and a lower fluorescent signal than obtained with 514 nm excitation. Alternatively, when excited by the 568 nm spectral line of the Ar—Kr laser found in some confocal laser-scanning microscopes, carboxy SNARF-1 exhibits a fluorescence increase at 640 nm as the pH increases and an emission isosbestic point at 585 nm. As with other ion indicators, intracellular environments may cause significant changes to both the spectral properties and pKa of carboxy SNARF-1, and the indicator should always be calibrated in the system under study.

When monitoring metabolic responses of cells, e.g., the rate of extracellular acidification, that are affected by the release of a gas from the cells, e.g., the release of CO₂, gas-permeable substrates are to be avoided in fabrication of microfluidic cell-retaining devices. For example, the soft polymer PDMS, or other highly gas-permeable polymers are preferably avoided. Instead, glass, silica, or metallized polymers are preferred. Thermosetting plastics, e.g. polystyrene, may also be employed with satisfactory result. Such plastic devices are particularly readily made by hot embossing, or other molding methods, as are well known in the prior art.

Monitoring Cellular Metabolism during Intermittent Flow:

After the cells are loaded, the rates of cell metabolism may be monitored by turning on the light excitation source (for fluorescence measurements) at the cell intersection points 14 and collecting light emitted from the intersection points 14. The excitation may be directed to the intersection points 14 either from the cell duct side of the porous membrane 20 or from the flow channel side of the porous membrane 20. Generally, excitation will be from the cell duct side if the indicator fluorophores are present either within the cells, or within particles trapped together with the cells. Alternatively, excitation generally will be from the flow channel side if the indicator fluorophores are present either as soluble fluorescent probes within the flow channels 12 or attached to particles present within the flow channels 12.

The flow in flow channels 12 may then cycled between the flowing conditions by creating a pressure gradient along channels 12. The pressure gradient is created by applying either pressure or vacuum to external channel openings 16 or 18. The pressure (negative or positive) may be small, e.g., between 0.1 psi and 3.0 psi, to reduce gas bubble formation. The flow will be turned on for a predetermined period. This “on period” is selected to match the time resolution desired for the measurements. Generally, for high resolution of time, the flow will be turned on only briefly, from between 1 second and 1 minute in order to achieve high time resolution. When less resolution is desired, however, the flow may be maintained on for longer periods of time, for example 1 hour to 24 hours. The rate of metabolic change (i.e., the slope in the change of a measured metabolic parameter vs. time) may be measured as signal during the period the flow is turned off. The “off period” may be predetermined, generally selected to be a short interval, also between 1 second and 1 minute for high time resolution. In any case, the “off period” should be great enough so that measured metabolic rates of the cells are high enough to be measured with precision. The precision in measuring metabolic rates during each “off period” may be between 0.1% and 5% coefficient of variation.

In a preferred mode of operation for pH monitoring, carboxy SNARF-1 is incorporated into the nutrient medium supplied in flow channels 12 at a concentration of about 1 micromolar. The cell medium is RPMI medium (without bicarbonate, without phenol red) but with 1 mM HEPES buffer, pH 7.2. The SNARF-1 fluorescence is excited at 488 nm by means of an argon ion laser, or alternatively with a xenon flash lamp fitted with optical filters to provide for a 10 nm excitation bandwidth. Excitation light enters the microfluidic cell-retaining device 2 through an optically transparent flow channel plate 42 within channel intersection regions 14 (where cells are retained at the membrane interface separating cell ducts 6 from flow channels 12). Fluorescent light emission also is collected through the optically transparent flow channel plate 42. The angles of excitation and collection of light emission optionally may be adjusted so as to avoid collection of specularly reflected excitation light, thereby decreasing the amount of stray excitation light detected as fluorescence.

The ratio of SNARF-1 emission at 570 nm and 640 nm may be measured by employing an optical dichroic beam splitter centered at 610 nm to split the emitted fluorescent light into two optical channels. A first optical emission channel has an optical interference filter centered at 570 nm and a second optical emission channel has an optical interference filter centered at 640 nm. Each of the optical filters has about a 10 nm band pass characteristic. The light passing through the optical filter in each channel is detected by means of a photodiode photodetector, or optionally a photomultiplier tube. The signal from each photodetector is converted to a voltage that is amplified by means of an electronic circuit. The voltage is then converted to a digital signal by an A/D converter and sent to a microprocessor where the relative ratio of 570 and 640 nm light emission is monitored over time. The change in this ratio over time indicates a change in the pH of the media in contact with the retained cells. An increase in the 570/640 ratio indicates acidification of the media, whereas a decrease in the ratio indicates an alkaline change in the medium pH. The pH change of the media may be determined by calibration of the system employing media of known pH to perform the calibration.

In an alternative embodiment, particles (with attached fluorescent reporter probes) may be used instead of soluble fluorescent probes. If fluorescent indicator particles are used to monitor the metabolism of the cells, the particles may be retained within the ducts and retained in contact with the cells. Alternatively, the particles may be maintained within diffusive communication of the cells, but supplied within the channels, together with fresh nutrient medium. Diffusive communication with the cells is achieved specifically at the junctions of the ducts and channels. The fluorescence of either soluble probes or particles may be monitored by excitation of the fluorescence either at the junctions of the ducts and channels, or alternatively within the channels downstream (i.e., distil) to an intersection of a duct and channel. The amplitude of the fluorescence, ratio of fluorescence, or other measured parameter related to cell metabolism is referred to as the measured “signal.”

Operation of a Valve-Less Cell-Retaining Fluidic Device:

In a preferred method of operation of the cell-retaining fluidic device, valves are not required. Thus, the device 2 does not require valves 50, 52, 54, nor valve channels 60, 62, 64, nor external openings 66, 68, 70, nor any additional pressure controllers 82, nor connectors 84 to control the valves. Thus, these structures may be omitted with considerable simplification to the microfluidic device, as well as to the peripheral apparatus, including pressure controllers, cartridge, and connectors.

In a preferred method of operation the following modified procedure may be used in cell loading and metabolism monitoring:

Cell-Loading:

Cell loading may be carried out as described above, except that in a preferred procedure, at least 50% of the volume of the agarose/cell suspension may be made up of agarose beads that are porous to liquid flow. The remaining volume of the suspension may be made up of the biological cells. Preferably from 80%-99% of the suspension volume may be made up of the porous agarose, so that the cells will not interfere with liquid flow through the cell ducts 6 at the intersection regions 14. The volume of agarose and cells that may be used to fill the intersection regions 14 may be carefully determined so that the intersection regions are not overly filled with the agarose/cell suspension. Next, a much less porous agar or gel, for example gelatin, acrylamide, or agar-agar, is added to the external wells of the cell ducts. Optionally, the less porous agar or gel may be drawn into the cell ducts 6 to the intersection regions 14 by again applying vacuum to external channel openings 14 and 16. A low porosity agar or gel with a melting temperature of 37-42 degrees centigrade may be heated to above the melting temperature to facilitate filling and subsequent gelation within the wells and cell ducts 6 when the temperature is reduced, (e.g., to room temperature, or otherwise less than 37 degrees centigrade.

The low porosity gel left in the cell ducts 6 may effectively function to prevent fluid from passing through the ducts, thus acting as a closed valve. Thus, the valves 54 in the cell ducts 6 may no longer be needed.

Monitoring Cellular Metabolism in Valve-Less Operation:

In a preferred method of operation without valves, the rates of cell metabolism are again monitored by turning on a light excitation source (for fluorescence measurements) at the cell intersection points 14 and collecting light emitted, as may be done in the method with valve structures given above. In this method, however, the flow in flow channels 12 may not be stopped completely during cycling of flow conditions. Instead, the flow rate is modulated in a cyclic fashion between a “rapid flow” state and a “slower flow” state. Since it is not necessary to stop the flow completely, valves 50 and 52 in flow channels 12 may not be required. As previously described, modulation of the pressure gradient may be created by applying either pressure or vacuum to external channel openings 16, 18. The pressure (negative or positive) may be moderate, even in the “rapid flow” condition, e.g., between 0.2 psi and 3.0 psi, to reduce gas bubble formation. The “rapid flow” condition may be turned on for a predetermined period of time. Next, modulation of the flow to the “slower flow” condition may be accomplished by reducing the applied pressure for a predetermined period of time. Generally the pressure (or vacuum) setting in the “slower flow” condition may be 50% or less than that used in the “rapid flow” condition. The ratio of pressures used in the “slower flow” condition and in the “rapid flow” condition is referred to as the “pressure modulation.” In a preferred mode of operation, 0.2 psi pressure may be used in the “slower flow” state and 2 psi pressure may be used in the “rapid flow” state. The pressure modulation in this case is a factor of 10.

The fraction of total time spent in the “slower flow” state may be referred to as the “duty cycle.” The duty cycle may be varied through a wide range, for example from 0.01 to 0.99. Generally, however, the duty cycle may be close to equal at 0.5. The cycle frequency (alternating from the “slower flow” state to the “rapid flow” state) may be pre-selected to match the time resolution desired for the measurements. The cycle frequency may be from between 1 second and 1 minute to achieve optimal time resolution, although cycle frequencies as short as 0.1 second or as long as 100 minutes, may be used in special cases. The cycle period should be great enough so that measured metabolic rates of the cells are high enough to be measured with precision. The precision in measuring metabolic rates during the “slower flow” state and in the “rapid flow” state within each cycle generally will be between 0.1% and 5% coefficient of variation.

The chemical indicators for monitoring the rates of metabolism in preferred, valve-less operating mode may be similar to those described above for operation in the mode requiring valves for starting and stopping of the flow. The rates of metabolism of the cells retained at the intersection region 14 of the device may be observed as a change in signal modulation over a pressure-modulation cycle, given constant conditions of:

a) Media buffering capacity,

b) Pressure modulation,

c) Duty cycle, and

d) Cycle frequency.

The change in signal observed over pressure-modulation cycles can be selected from any number of conveniently measured parameters. For example the peak-to-peak (or RMS) amplitude of fluorescence changes may be measured. Alternatively, the rate of change in fluorescence upon switching from either the “rapid flow” to the “slower flow” state (or vice-versa) may be selected as the parameter related to the rate of cell metabolism.

Modes of Characterization of Cell-Affecting Agents:

The above methods may be used to characterize the effects of any agent that may cause a change in the cellular metabolism monitored by the method. For example, introduction of a chemical, cell-affecting agent (that changes the metabolic rate) into the flow channels 12, so that cells retained at the intersection point 14 are exposed to the agent, will be observed as a change in metabolic rate observed prior to the introduction of the agent in comparison to the metabolic rate observed after introduction of the agent. The ratio of the rates prior and after introduction of the agent is an index of the effectiveness of the agent at altering cellular metabolism. This mode of operation may be especially useful for determining the effect of potential drug-candidates on cells (e.g., in drug-screening).

Another mode of characterization of cell-affecting agents includes introducing different agents (or different concentrations of the same agent) into different flow channels and monitoring the change in metabolic rates over time. For example, toxic agents may cause a more rapid decrease in metabolic activity in comparison to non-toxic agents.

Still another mode of characterization is to utilize different types of cells in the different channels of the cell-retaining device and to compare the changes of metabolic activity over time. For example, cells sampled from human patients may be tested for a selected physiological function by providing the cells in the cell retaining device and providing a stimulus of the physiological function to the cells. The rates of stimulation in metabolic rate of control cells (with the physiological function) are compared to cells with unknown function in order to diagnose the functional state of the cells. For example, cells with altered chloride channel activity (e.g. those present in human cells having cystic fibrosis genotype) can show altered metabolic activity (compared to a normal control) when the cells are exposed to a chloride channel stimulus, or agonist.

The cell-affecting agents tested may be chemical or physical, or the interaction between the agents. For example, the effect of temperature, light (or other irradiation) intensity or frequency may be measured. Chemical agents may be soluble molecules, e.g., small organic molecules, or may be macromolecules such as oligonucleotides (including DNA or RNA) peptides (including proteins), complex carbohydrates, lipids, or the like. The chemical agents also may be gases dissolved in the cell media. The chemical agents also may be fine solid particulates (e.g., 10 nm to 100 μm in diameter) that are provided in contact with the cells in the cell ducts (provided that the cell ducts are sufficiently wide and deep to accommodate the particles).

The invention may be embodied in other specific forms without departing from the spirit or essential characteristics thereof. The foregoing embodiments are therefore to be considered in all respects illustrative of the invention described herein. Various features and elements of the different embodiments can be used in different combinations and permutations, as will be apparent to those skilled in the art. Scope of the invention is thus indicated by the appended claims rather than by the foregoing description, and all changes which come within the meaning and range of equivalency of the claims are therefore intended to be embraced herein.

I claim:

1. A method for maintaining cell viability in a microfluidic device, the method comprising the steps of:
providing a cell proximate a first side of a porous membrane of the microfluidic device; and
providing a media comprising a cell nutrient proximate a second side of the porous membrane, wherein the porous membrane is adapted to prevent the cell from passing therethrough, to substantially prevent the media from flowing therethrough, and to provide diffusive communication between the two sides to allow the cell nutrient and a cell product to pass therethrough.

2. The method of claim 1, further comprising the step of detecting the cell product in the media.

3. The method of claim 2, wherein detecting the cell product in the media comprises detecting at least one of an electrochemical signal and a luminescent emission.

4. The method of claim 1, wherein the porous membrane comprises a material selected from the group consisting of glass fiber, polycarbonate, polyethylene, polypropylene, polystyrene, polyimide, cellulose, nitrocellulose, cellulose esters, nylon, rayon, fluorocarbon, perfluorocarbon, polydimethylsioloxane, polyester, acrylics, acrylonitrile-butadiene-styrene; polyoxy-methylene; polyarylate, polyvinylchloride, PBT-polyester, polybenzimidazone, acetal copolymers, polyimides, ethylene-chlorotrifluorethylene, PET polyesters, ethylene-tetrafluorethylene, fluorinated ethylene propylene, polyphenylene sulfide, polyethylene, polyurathanes, polyketones, polychloro-trifluoro-ethylene, polyvinylidene fluoride, polyethylene terephthalate polyesters, polypropylene oxides, polypropylene styrenes, polyether-ether ketones, polytetrafluorethylene, polyarylether sulfones, polyamide-imides, polyphenylene sulfides, polyarylates, polymethylpentene, polyketones, polysulfones, polyphenylene sulfides, PBT polyesters, and/or alloys of polymers.

5. The method of claim 1, wherein the step of providing the media comprises flowing the media along at least a portion of the second side of the porous membrane.

6. The method of claim 5, wherein flowing the media comprises intermittently flowing the media.

7. The method of claim 1, further comprising the step of controlling a temperature of the cell.

8. The method of claim 1, further comprising the step of controlling a concentration of the cell nutrient in the media.

9. A method for loading cells into a microfluidic device, the method comprising the steps of:
depositing a cell sample into a common duct opening of the microfluidic device; and
subdividing the cell sample, so that at least a first portion of the sample flows into a first cell duct in fluidic communication with the duct opening and another portion of the sample flows into a second cell duct in fluidic communication with the duct opening.

10. The method of claim 9, wherein the step of subdividing the cell sample comprises flowing at least a portion of the cell sample through a manifold interdisposed between the duct opening and at least one cell duct.

11. The method of claim 9, wherein at least one of the sample portions flows by capillary action.

12. The method of claim 9, wherein the step of subdividing the cell sample comprises substantially uniformly dividing the cell sample.

13. The method of claim 9, wherein the step of subdividing the cell sample includes applying a pressure differential.

14. The method of claim 9, wherein the cell sample comprises a substantially isopycnic solution having a density substantially similar to a density of cells in the sample, such that the cells remain substantially in neutral suspension in the isopycnic solution.

15. A microfluidic device for maintaining viability of a cell, the device comprising:
a cell duct plate, defining at least one cell duct therein;
a porous membrane having a first side bounding at least a portion of the cell duct; and
a flow channel plate, defining at least one flow channel therein, at least a portion of the flow channel being bounded by a second side of the porous membrane, wherein the cell duct and the flow channel are in diffusive communication through the membrane and the porous membrane is adapted to prevent a cell in the cell duct from passing therethrough, while allowing a cell nutrient in the flow channel and a cell product in the cell duct to pass therethrough.

16. The microfluidic device of claim 15, wherein the cell duct plate comprises a material selected from the group consisting of glass, fused silica, quartz, silicon, and organic polymers.

17. The microfluidic device of claim 15, wherein the flow channel plate comprises a material selected from the group consisting of glass, fused silica, quartz, silicon, and organic polymers.

18. The microfluidic device of claim 15, wherein the porous membrane comprises a material selected from the group consisting of glass fiber, polycarbonate, polyethylene, polypropylene, polystyrene, polyimide, cellulose, nitrocellulose, cellulose esters, nylon, rayon, fluorocarbons, perfluorocarbons, polydimethylsiloxane, polyester, acrylics, acrylonitrile-butadiene-styrene; polyoxy-methylene; polyarylate, polyvinylchloride, PBT-Polyester, polybenzimidazone, acetal copolymers, polyimides, ethylene-chlorotrifluorethylene, PET polyesters, ethylene-tetrafluorethylene, fluorinated ethylene propylene, polyphenylene sulfide, polyethylene, polyurathanes, polyketones, polychloro-trifluoro-ethylene, polyvinylidene fluoride, polyethylene terephthalate polyesters, polypropylene oxides, polypropylene styrenes, polyether-ether ketones, polytetrafluorethylene, polyarylether sulfones, polyamide-imides, polyphenylene sulfides, polyarylates, polymethylpentene, polyketones, polysulfones, polyphenylene sulfides, PBT polyesters, and alloys of polymers.

19. The microfluidic device of claim 15, wherein the porous membrane defines a pore size having a diameter selected from the range of about 1 nanometer to about 100 micrometers.

20. The microfluidic device of claim 15, wherein the porous membrane has a thickness less than about 200 microns.

21. The microfluidic device of claim 20, wherein the thickness is greater than about 5 microns.

22. The microfluidic device of claim 15, wherein the porous membrane comprises an interfacial layer disposed between the cell duct plate and the flow channel plate.

23. The microfluidic device of claim 15, further comprising a plurality of cell ducts in combination with a plurality of flow channels.

24. The microfluidic device of claim 23, wherein a number of cell ducts is equal to a number of flow channels.

25. The microfluidic device of claim 23, wherein the cell ducts are generally radially disposed about a common duct opening.

26. The microfluidic device of claim 23, wherein at least two flow channels are not in mixing fluidic communication with each other.

27. The microfluidic device of claim 23, wherein at least two flow channels are in mixing fluidic communication with each other.

28. The microfluidic device of claim 15, wherein at least one of a cell duct and a flow channel further comprises a valve.

29. A microfluidic device for retaining a cell sample including a plurality of cells, the device comprising:
a plate defining:
a common duct opening adapted to receive the cell sample; and
at least two cell ducts in fluidic communication with the duct opening, so that at least a portion of the cell sample can flow into a first cell duct and another portion of the cell sample can flow into a second cell duct.

30. The microfluidic device of claim 29, wherein the plate further defines a manifold interdisposed between the duct opening and at least one cell duct.

31. The microfluidic device of claim 29, further comprising a pressure differential source adapted to induce the flow of at least one of the cell sample portions into at least one of the cell ducts.

32. A system for monitoring an activity of a cell, the system comprising:
a microfluidic device comprising:
a cell duct plate defining at least one cell duct therein;
a porous membrane having a first side bounding at least a portion of the cell duct; and
a flow channel plate, defining at least one flow channel therein, at least a portion of the flow channel being bounded by a second side of the porous membrane, wherein the porous membrane is adapted to prevent a cell in the cell duct from passing therethrough, while allowing a nutrient in the flow channel to pass therethrough and allowing a product of the cell to pass therethrough;
a pump adapted to induce flow of a nutrient media through the flow channel to support cell viability in the cell duct;
a controller adapted to control flow in the microfluidic device; and
a sensor adapted to detect at least one of the cell and the product of the cell.

33. The system of claim 32, wherein the sensor comprises at least one of an electrochemical detector and a luminescence detector.

34. The system of claim 33, wherein the luminescence detector comprises a fluorescent reagent, an excitation light source adapted to provide radiation having a first radiation wavelength range, and a detector adapted to measure an intensity of emitted light in a second radiation wavelength range, the second radiation wavelength range being different from the first radiation wavelength.

35. The system of claim 33, wherein the electrochemical detector comprises an electrode adapted to measure at least one of pH and dissolved oxygen.
